(19) United States
(12) Patent Application Publication
(10) Pub. No.: US 2005/0123932 A1
(43) Pub. Date: Jun. 9, 2005

(54) Nucleic acid-chelating agent conjugates

(76) Inventor: Mekbib Astatke, Germantown, MD (US)

Correspondence Address: BANNER & WITCOFF, 1001 G STREET N W, SUITE 1100, WASHINGTON, DC, 20001, US

(21) Appl. No.: 10/729,898

(22) Filed: Dec. 9, 2003

(51) Int. Cl. C12Q 1/68; C07H 21/04; C12P 19/34; C07K 14/00

(52) U.S. Cl. 435/6; 435/91.2; 536/25.32; 530/395

(57) ABSTRACT

A nucleotide having covalently bonded thereto a chelating agent can be used by a nucleic acid polymerase to synthesize a nucleic acid-chelating agent conjugate. The nucleic acid-chelating agent conjugate can chelate a transition metal ion and be used to detect a polyhistidine-containing recombinant protein.

### FIELD OF THE INVENTION

The invention relates to the field of nucleic acids. More specifically, it relates to nucleic acid-chelating agent conjugates. The invention also relates to compositions and nucleotide triphosphates for carrying out the synthesis of nucleic acids of the invention.

### BACKGROUND OF THE INVENTION

Expression of recombinant proteins is very common in molecular biology today. It is common to express a recombinant protein with a variety of different tags, for example a GST tag or a polyhistidine tag. (Smith D B, Johnson K S (1988) Gene 670:3140; U.S. Pat. No. 5,284,933; and U.S. Pat. No. 5,310,663).

A polyhistidine tag (His-tag) is added to a recombinant protein to aid in purification of the protein. Polyhistidine sequences can coordinately bind a transition metal ion, such as nickel. A chelating agent is typically covalently bonded to an agarose bead and used to create a column for purifying polyhistidine-containing proteins. (U.S. Pat. No. 4,569,794, U.S. Pat. No. 5,047,513, U.S. Pat. No. 6,242,581, U.S. Pat. No. 6,479,300, and Prath, J et al. (1975) Nature 258, 598-599).

The polyhistidine tag also offers a convenient tag for detecting the recombinant protein in an ELISA assay, immunohistochemical staining assay, or an immunoblot assay. The polyhistidine sequence can be detected with the use of an anti-histidine tag antibody or an enzyme-chelating agent bound to a nickel ion. The anti-histidine tag antibody is typically detected using an anti-antibody coupled to an enzyme. Once the antibody or enzyme-chelating agent-nickel ion conjugate is bound, the assay is developed using a substrate for the enzyme. (U.S. Pat. No. 5,840,834, Hochuli, E. and Piesecki, S. (1992) A companion to Method in Enzymology 4, 68-72, and Lindner, P et al., (1997) BioTechniques 22, 140-149).

Enzyme conjugates, whether they are antibody-enzyme conjugates or enzyme-chelating agent-nickel conjugates, are typically unstable. The enzymes become degraded over time and are thus less effective in detecting the polyhistidine-containing recombinant protein.

Labels have been attached to a nucleic acid. Storage of a labeled nucleic acid is typically not practiced because the labels can be added to the nucleic acid prior to use.

### BRIEF SUMMARY OF THE INVENTION

This invention provides a nucleic acid having covalently bonded to at least one nucleotide of the nucleic acid a chelating agent.

This invention also provides a nucleotide conjugated to a chelating agent.

In one embodiment of the invention a nucleic acid having covalently bonded to at least one nucleotide of the nucleic acid a chelating agent is provided. The covalently bonded chelating agent has an affinity for a transition metal ion.

In another embodiment of the invention a method of generating a nucleic acid having covalently bonded to at least one nucleotide of the nucleic acid a chelating agent is provided. The covalently bonded chelating agent has an affinity for a transition metal ion. The first step is to determine which nucleotides in the nucleic acid will be covalently bonded to the chelating agent. The second step is to synthesize the nucleic acid utilizing a nucleotide having covalently bonded thereto a chelating agent determined in the first step.

In still another embodiment of the invention a method of generating a nucleic acid having covalently bonded to at least one nucleotide of the nucleic acid a chelating agent is provided. The covalently bonded chelating agent has an affinity for a transition metal ion. The nucleic acid is provided and the chelating agent is bonded to the nucleic acid with a crosslinking agent.

In still yet another embodiment a nucleotide-chelating agent conjugate is provided. The nucleotide-chelating agent conjugate has a nucleotide covalently bonded thereto a chelating agent. The covalently bonded chelating agent has an affinity for a transition metal ion.

In another embodiment of the invention a method of synthesizing a nucleotide-chelating agent conjugate is provided. The method comprises the step of covalently bonding a chelating agent to a nucleotide to form the nucleotide-chelating agent conjugate. The covalently bonded chelating agent has an affinity for a transition metal ion.

In still another embodiment of the invention a method of chelating a transition metal ion to a nucleic acid is provided. The nucleic acid has a chelating agent covalently bonded to at least one nucleotide of the nucleic acid. The covalently bonded chelating agent has an affinity for a transition metal ion. The first step of the method is to mix an excess of the transition metal ion and the nucleic acid to form a mixture. The second step is to incubate the mixture for a time to form a transition metal-chelating agent-nucleic acid chelate. The third step is to purify the transition metal-chelating agent-nucleic acid chelate from the excess transition metal ion.

In still yet another embodiment of the invention a method for detecting a polyhistidine-containing recombinant protein is provided. The first step is to form a conjugate of a transition metal-chelating agent-nucleic acid chelate with a polyhistidine-containing recombinant protein. The second step is to detect the so-formed conjugate.

In another embodiment of the invention a method for His-tag amplification of a transition metal-chelating agent-nucleic acid chelate is provided. The method comprises the step of amplifying the nucleic acid portion of the chelate.

In still another embodiment of the invention a method for identifying a peptide ligand that binds to a biomolecule is provided. The peptide is identified from a peptide library. The method comprises the steps of immobilizing the biomolecule, contacting the biomolecule with a peptide library, forming a conjugate of a transition metal ion-chelating agent-nucleic acid chelate with the polyhistidine sequence, and detecting the chelate. The peptide library comprises peptides having a polyhistidine sequence.

In still yet another embodiment of the invention a method for identifying a biomolecule that can bind to a peptide ligand is identified. The method comprises the steps of providing a biomolecule mixture, resolving the biomolecule mixture, immobilizing the biomolecule mixture, contacting the biomolecule mixture with a peptide library, forming a conjugate of a transition metal ion-chelating agent-nucleic acid with the polyhistidine sequence of the peptides, and detecting the chelate. The peptide library comprises peptides having a polyhistidine sequence.

In another embodiment of the invention a method for identifying a biomolecule that can bind to a peptide ligand is provided. The method comprises the steps of providing a biomolecule mixture, contacting the biomolecule with a peptide library, resolving the biomolecule mixture, immobilizing the biomolecule mixture, forming a conjugate of a transition metal ion-chelating agent-nucleic acid chelate with the polyhistidine of the peptides, and detecting the chelate. The peptide library comprises peptides having a polyhistidine sequence.

The invention thus provides the art with a nucleic acid comprising a chelating agent bonded to at least one nucleotide of the nucleic acid.

### BRIEF DESCRIPTION OF THE DRAWINGS

FIG. 1A shows substrates A and B used for a nucleic acid synthesis reaction. Each substrate contains a 20-mer primer (SEQ ID NO:1) annealed to a 34-mer (SEQ ID NO:3) or a 35-mer (SEQ ID NO:2) template.

FIG. 1B shows the synthesized nucleic acid denoted as Probes A and B. The nucleotides covalently bonded to a chelating agent are shown. The chelating agent is α-N,N-bis-carboxymethyl lysine (CM-Lys).

FIG. 2A shows a gel assay to verify synthesis of nucleic acid using substrate A. Panels I and II indicate DNA polymerase reactions catalyzed by the Klenow fragment of E. coli. DNA polymerase (37° C.) and Taq DNA polymerase (70° C.), respectively. The unextended labeled primer is denoted “P” and was loaded in the “substrate” lane of the gel. The full-length synthesized nucleic acid is denoted “F.L.” The elongation reactions were performed under different nucleotide triphosphate combinations: where the reaction denoted by lane ‘a’ was in the presence of the 4 dNTPs at 100 μM each; lanes ‘b’ and ‘c’ represent extensions in the presence of only 3 dNTPs (dTTP, dGTP & dATP) at 100 μM of each and lanes ‘d’ and ‘e’ represent extension reactions in the presence of dCTP-CM-Lys (200 μM), and dTTP, dGTP and DATP at 100 μM of each. The polymerase reaction times were 5 minutes for those represented by lanes ‘a’, ‘b’ and ‘d’, and 1 hour represented by lanes ‘c’ and ‘e’. The DNA ladder denoted as T, C, G and A represent the presence of ddTTP, ddCTP, ddGTP and ddATP for the respective lane in the polymerase reaction catalyzed by the exonuclease deficient derivative of the T7 DNA polymerase.

FIG. 2B shows a gel assay to verify synthesis of nucleic acid using substrate B. Panels I and II are as described above for FIG. 2A except the DNA ladder represents C (ddCTP) and A (ddATP).

FIG. 3 shows the detection of polyhistidine-containing recombinant proteins (β-gal (His)₆ and BLV-I (His)₆) with a nucleic acid-chelating agent conjugate of the invention. Wild-type β-gal is a negative control. Lanes ‘a’, ‘b’, ‘c’, ‘d’ and ‘e’ represent 2 μg, 400 ng, 80 ng, 16 ng and 3.2 ng of β-gal (His)₆ protein, respectively. Lanes ‘f’, ‘g’, ‘h’, ‘i’ and ‘j’ represent 2 μg, 400 ng, 80 ng, 16 ng and 3.2 ng of BLV-I (His)₆) protein, respectively. Lanes “X” and “Y” represent wild-type β-gal protein. The lane labeled as “L” denotes a standard His-tag protein ladder purchased from Qiagen, with the corresponding kDa.

### DETAILED DESCRIPTION OF THE INVENTION

It is a discovery of the present inventors that a chelating agent can be conjugated to a nucleic acid, chelated to a transition metal ion, and used to detect polyhistidine-containing recombinant proteins.

Nucleic Acid-Chelating Agent Conjugate

The nucleic acid-chelating agent conjugate of the present invention has covalently bonded to at least one nucleotide of the nucleic acid, a chelating agent. The covalently bonded chelating agent has an affinity for a transition metal ion.

The nucleic acid can be deoxyribonucleic acid (DNA) or ribonucleic acid (RNA), and can be single-stranded (ss), double stranded (ds), or a hybrid of RNA and DNA. Nucleic acid derivatives may also be used such as protein nucleic acid (PNA) and locked nucleic acid (LNA) and nucleic acid molecules comprising modified nucleotides. The nucleic acid can be a single base to several bases to several thousand bases in length. For example, the nucleic acid can be 1, 5, 10, 15, 20, 30, 50, 100, 500, 1000, or more bases in length.

Chelating Agents

Chelating agents have an affinity for a transition metal ion, and thus, any chelating agent that coordinately chelates a transition metal ion with polyhistidine can be used in the practice of the present invention. For example, nitrilotriacetic acid (NTA) can be used. Other suitable chelating agents include but are not limited to iminodiacetic acid (IDA), bicinchoninic acid (BCA) or N,N,N′-tris(carboxymethyl)ethylenediamine (TED). Preferably the chelating agent is NTA. The NTA chelating agent can be synthesized, for example, using published methods (see, for example, U.S. Pat. No. 4,877,830) from an epsilon amino blocked lysine residue. For example, the blocked lysine residue can be reacted with bromoacetate as shown below.

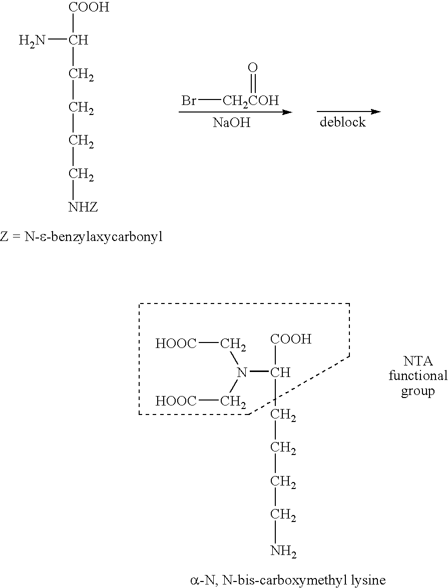

The reaction forms α-N,N-bis-carboxymethyl lysine (CM-Lys), which is an exemplary chelating agent for the practice of the invention. Other methods commonly known in the art can be utilized to synthesize the chelating agent. For example, a chelating agent discussed above can be added to tyrosine or cysteine.

Method of Generating a Nucleic Acid-Chelating Agent Conjugate

A nucleotide-chelating agent conjugate can be incorporated into a nucleic acid through a nucleic acid synthesis reaction. The location of the nucleotide-chelating agent conjugate can be determined by the skilled artisan by reviewing the sequence of the nucleic acid to be synthesized. The skilled artisan determines which nucleotide or nucleotides will be covalently bonded to the chelating agent and the nucleic acid is synthesized using well-known methods in the art. For example, a nucleic acid synthesis reaction can be an enzymatic reaction or a chemical reaction. Enzymatic reactions typically use a DNA polymerase, a PCR polymerase, an RNA polymerase, a reverse transcriptase, or mutants, variants, or derivatives thereof. The DNA polymerases include a DNA polymerase derived from a mesophilic organism (i.e., an organism that has an optimal growth temperature of 25° C. to 40° C.), such as, for example, E. coli DNA polymerase I (proficient or deficient in 3′→5′ exonuclease activity), T4 DNA polymerase, or mutants, variants, or derivatives thereof. The PCR polymerases include, for example thermostable polymerases, such as, Taq, Tne, Tma, Tth, Pfu, VENT™, DEEPVENT™, Pfx™, or mutants, variants, or derivatives thereof. The RNA polymerases include, for example, SP6, T7, T3, or mutants, variants, or derivatives thereof. The reverse transcriptases include, for example, AMV, MMLV, SuperScriptII™, or mutants, variants, or derivatives thereof. Examples of nucleic acid synthesis reactions include, but are not limited to DNA polymerase fill-in reactions, PCR, reverse transcription, terminal transferase, and RNA transcription reactions, and chemical oligonucleotide synthesis reactions.

Alternatively, the chelating agent can be attached directly to a nucleic acid with a crosslinking agent. The chelating agent can be added to the nucleic acid using, for example, a crosslinking reaction (e.g., maleimide). Crosslinking reactions are well known in the art. The chelating agent can also be added to the nucleic acid though a modification of a nucleotide, for example, with a succinimidyl ester. The chelating agent can subsequently be covalently bonded to the nucleic acid by reaction with the crosslinking agent.

Nucleic Acid Synthesis Reactions

The nucleic acid synthesis reactions use a nucleotide mixture containing all the nucleotides to synthesize the nucleic acid. However, one or more nucleotide types can be substituted partially or wholly with a nucleotide-chelating agent conjugate (i.e., a nucleotide covalently bonded to a chelating agent). The nucleotides can be a deoxyribonucleotide or a ribonucleotide or derivative thereof. The nucleotides can be in a mono-, di-, or triphosphate form. Preferably the nucleotides are in the triphosphate form. If the nucleotides are in the mono- or diphosphate form then the mono- or diphosphate nucleotides are preferably converted to the triphosphate form by methods well known in the art. For example, a mono- or diphosphate nucleotide can be converted to the triphosphate form by a nucleoside monophosphate kinase and a nucleoside diphosphate kinase.

The deoxyribonucleotide can be any deoxyribonucleotide or a derivative or analog of any deoxynucleotide. For example, the deoxyribonucleotide can be deoxyadenosine (dA), deoxycytidine (dC), deoxyguanosine (dG), deoxythymidine (dT), or deoxyinosine (dl). Thus, for example, the deoxynucleotide can be deoxycytidine triphosphate (dCTP), deoxycytidine diphosphate (dCDP), or deoxycytidine monophosphate (dCMP). Examples of analogs include, but are not limited to, dATPaS, dCTPaS, and 5-methyl-dCTP. Examples of derivatives include, but are not limited to, biotinylated-dATP, biotinylated-dCTP, biotinylated-dGTP, biotinylated dTTP, fluorescein-dATP, fluorescein-dCTP, fluorescein-dGTP, fluorescein-dTTP, rhodamine-dATP, rhodamine-dCTP, rhodamine-dGTP, rhodamine-dTTP, and Cy5-dCTP

The ribonucleotide can be any ribonucleotide or a derivative or analog of any ribonucleotide. For example, the ribonucleotides can be adenosine (A), cytidine (C), guanosine (G), or uracil (U). Thus, for example, the ribonucleotides can be cytidine triphosphate (CTP), cytidine diphosphate (CDP), or cytidine monophosphate (CMP). Examples of analogs include, but are not limited to, 3′-O-methyl-GTP, 7-methyl-GTP, 2-O-methyl-ATP, 2-O-CTP, 2-O-GTP, and 2-O-UTP. Examples of derivatives include, but are not limited to, biotinylated-ATP, biotinylated-CTP, biotinylated-GTP, and biotinylated-UTP, fluorescein-ATP, fluorescein-CTP, fluorescein-GTP, fluorescein-UTP, rhodamine-ATP, rhodamine-CTP, rhodamine-GTP, and rhodamine-UTP.

Fill-In Reactions

A DNA polymerase or a PCR polymerase can be used to fill in a 5′ overhang (Kornberg A. and Baker T. A. (1992) DNA replication (Freeman, San Fransisco)). If the nucleic acid is, for example, a restriction fragment then a nucleic acid-chelating agent conjugate can be generated using a fill-in reaction. If the 5′ overhang is small, for example less than 10 nucleotides (i.e., 10, 9, 8, 7, 6, 5, 4, 3, 2, or 1), the fill-in reaction can use a nucleotide mixture that has a combination of nucleotide-chelating agent conjugates. That is, the mixture might, for example, contain dCTP-chelating agent and dGTP-chelating agent, or the mixture might contain dATP-chelating agent and dTTP-chelating agent. Using, for example, a nucleotide mixture that contains dCTP-chelating agent, dGTP-chelating agent, DATP and dTTP, then wherever a dC or dG is incorporated into the newly synthesized nucleic acid, a chelating agent may be present. Similarly, if the mixture contains dATP-chelating agent, dTTP-chelating agent, dCTP and dGTP, then wherever a dA or dT is incorporated into the newly synthesized nucleic acid, a chelating agent may be present.

Alternatively, the mixture can contain for a specific nucleotide a ratio of nucleotide and nucleotide-chelating agent conjugates. For example, the mixture might contain a 50:50 mixture of nucleotide-chelating agent conjugates to nucleotides. In this manner, the 5′ overhang will be filled in and less than all of the newly synthesized (i.e., filled in) nucleic acid will contain the chelating agent covalently bonded to the nucleic acid. The number of nucleotide-chelating agent conjugates incorporated into the newly synthesized DNA can be controlled by the ratio of nucleotide-chelating agent conjugates to nucleotides. The higher percentage of nucleotide-chelating agent conjugates, the more newly synthesized nucleic acid may contain the nucleotide-chelating agent conjugate.

### Fill-In Reaction for Annealed Oligonucleotides of Unequal Length

A DNA polymerase or a PCR polymerase can be used to fill-in a 5′ overhang created when two oligonucleotides of unequal length are annealed. The size of the region to be filled in and the sequence of the region to be filled in can be controlled by the length of the two oligonucleotides and the sequence chosen by the skilled artisan. See Example 2 below.

Once the oligonucleotides have been designed and synthesized, the oligonucleotides can be heated to remove any secondary structure and annealed to form double stranded nucleic acid with a 5′ overhang. Typically, the oligonucleotides are mixed and are heated to approximately 100° C. for about 1 minute and slowly cooled to allow double stranded oligonucleotides to form with their respective complement.

RNA Transcription

An RNA polymerase can be used to synthesize nucleic acid comprising at least one nucleotide covalently bonded to a chelating agent. A template sequence, usually DNA, can be provided which comprises an RNA polymerase start site and an RNA termination site. These sites can flank a sequence to be transcribed. RNA polymerase start sites are well known in the art and include, for example, a T7, SP6, and T3 RNA polymerase start sites. RNA polymerase stop sites are well known in the art and include, for example, a T7, SP6, and T3 stop sites. Alternatively, a stop site can be created in the template by cleaving the template at a point where transcription termination is desired. The starting template is preferably DNA, and the DNA template can be destroyed using, for example, a DNase. RNA polymerases are, for example, a T7, SP6, or T3 RNA polymerase.

Reverse Transcription

A reverse transcriptase can be used to synthesize a nucleic acid comprising at least one nucleotide covalently bonded to a chelating agent. Following cDNA synthesis, the RNA template can be destroyed. The RNA can be destroyed using, for example, an RNase (e.g., RNase H). Destruction of the RNA creates a single stranded DNA comprising at least one nucleotide covalently bonded to a chelating agent. Examples of reverse transcriptases include, but are not limited to AMV, MMLV, and Superscript II™ (Invitrogen, Carlsbad, Calif.).

Chemical Synthesis of a Nucleic Acid

A nucleic acid-chelating agent conjugate can be synthesized using, for example, phosphoroamidite chemistry. The nucleic acid-chelating agent conjugate can be synthesized using an automated oligonucleotide synthesizer (Caruthers M. H. Science (1985) 230: 281-285). A nucleotide-chelating agent conjugate can be substituted for a nucleotide in the synthesis reaction. Alternatively, the nucleic acid can be synthesized using the automated oligonucleotide synthesizer and the chelating agent added post nucleic acid synthesis using a chemical reaction. Use of the automated oligonucleotide synthesizer would allow easy incorporation of other modifications that would confer nuclease resistance to the nucleic acid. Nuclease resistance can be conferred, for example, by use of a phosphorothioate linkage, 2′-O methyl ribose, peptide-nucleic acid (PNA), and locked nucleotide acid (LNA) (Lammond A. I. and Sproat B. S. (1993) FEBS Lett. 325 (1-2) 123-7). Synthesizing the nucleic acid-chelating agent conjugate on an automated oligonucleotide synthesizer usually enables preparation of smaller size nucleic acids than an enzymatic preparation of a nucleic acid.

Crosslinking a Chelating Agent to a Nucleic Acid

### Conjugating a Chelating Agent to a Nucleic Acid Using a Crosslinker

A chelating agent can be added to a nucleic acid after synthesis of the nucleic acid. The nucleic acid can be prepared using amine-modified nucleotides at positions where a chelating agent is desired. Amine modified nucleotides include, but are not limited to amine modified dA, dC, and dT nucleotides. Post nucleic acid synthesis, a chelating agent can be added to the nucleic acid by crosslinking a chelating agent and the amine modified base or bases in the nucleic acid using a crosslinking agent. Typically, the nucleic acid is incubated with, for example, an NHS-ester-maleimide heterobifunctional crosslinking agent (Pierce Biotechnology, Rockford Ill.) in 0.1 M carbonate/bicarbonate buffer at pH 7.5 to form a nucleic acid-crosslinking agent conjugate. The molar ratio of nucleic acid to crosslinking reagent is typically 1 to 10. The reaction is typically incubated for about an hour at room temperature with gentle mixing. The nucleic acid can subsequently be precipitated by adding 3 volumes of 2% (by weight/volume) lithium perchlorate in acetone and pelleting for 5 minutes at 13,000 rpm. The nucleic acid pellet can be resuspended in 0.1 M carbonate/bicarbonate buffer at pH 7.5. The nucleic acid-crosslinking agent conjugate is typically mixed with a chelating agent in a molar ratio of about 1 to 20. The mixture can be incubated at room temperature for about an hour to form a nucleic acid-chelating agent conjugate. Following incubation, the nucleic acid-chelating agent conjugate can be purified with by eluting over a G-25 or a G-50 column or using HPLC.

Methods of Synthesizing a Nucleotide-Chelating Agent Conjugate

The nucleotide-chelating agent conjugate can be synthesized using an enzymatic reaction catalyzed, for example, by a nucleic acid modifying enzyme. Examples of nucleic acid modifying enzymes include, but are not limited to, pyrophosphatase, terminal nucleotidyl transferase, recombinase, ligase, isomerase, and a ribozyme.

Alternatively, the nucleotide-chelating agent conjugate can be synthesized using a chemical reaction, such as, for example, a transamination reaction or a crosslinking reaction. Draper (NAR 12:989-1002, 1984) describes transamination reactions for coupling reporter molecules to nucleotides. An exemplary method for bonding a chelating agent and a nucleotide is the following transamination reaction.

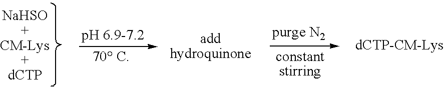

The reaction product, i.e., the dCTP-CM-Lys, can be monitored, for example, using HPLC and mass spectrometry. For example, formation of dCTP-CM-Lys can be monitored by HPLC using a C-18 column. Solvents for the HPLC can be, for example, 5 mM tetrabutyl ammonium phosphate (TBAP) in 60 mM NH₄H₂PO₄ at pH 5 for solvent A and 5 mM TBAP in methanol for solvent B.

Methods of Purifying the Nucleotide-Chelating Agent Conjugate

The nucleotide-chelating agent conjugate can be purified by any method known in the art for purifying a nucleotide. For example, the nucleotide-chelating agent conjugate can be purified over a DEAE-Sephadex A-25 column using an ionic gradient of 0.1 M to 1 M triethylammonium bicarbonate buffer pH 7.0-7.5. Fractions can be collected and pooled. The pooled fractions can be dried, for example, using a Rotovapor and washed with ethanol. The pooled fractions can be resuspended and the purity of the fractions can be quantitated, for example, by HPLC using a C-18 column. Solvents for the HPLC can be those described above.

Number of Chelating Agents Present in Nucleic Acid

The number of chelating agents present in a nucleic acid can be at least one (e.g., 1, 2, 3, 4, or 5 or more). Preferably, the number of chelating agents is greater than 5 (e.g., 5, 6, 7, 8, 9, 10, 12, 15, 17, 20, 25, 50, 75, 100, 250, 500, or 1000 or more), and possibly greater than 10 per nucleic acid. The number can be greater than 20, greater than 50, or greater than 100 chelating agents per nucleic acid. The size of the nucleic acid to be synthesized and the sequence determine the greatest number of chelating agents present in the nucleic acid. For example, if the nucleic acid-chelating agent conjugate is generated by a fill-in reaction from a 5′ overhang and the 5′ overhang is six nucleotides long, then the maximum number of chelating agents in the nucleic acid is six. One skilled in the art will also recognize that some polymerases possess terminal nucleotidyl transferase activity and can add non-template directed nucleotides to the end of the nucleic acid. Terminal nucleotidyl transferase activity can result in, for example, n+1 or n+2 products. The terminally transferred nucleotides can remain or can be removed with an exonuclease, for example E. coli exonuclease VII. If the terminally transferred nucleotides remain, the skilled artisan will know that the total number of chelating agents can be greater than that calculated by the size of the 5′ overhang.

However, if the nucleic acid-chelating agent conjugate is generated by an RNA polymerase reaction and the RNA transcript is 100 nucleotides in length then the maximum number of chelating agents is 100. If that same RNA transcript contains 20 cytidine residues and the nucleotide mixture contains CTP-chelating agent conjugate, ATP, GTP, and UTP, then the maximum number of chelating agents in the nucleic acid is 20 assuming the fidelity of the polymerase to be 100%.

Example 2, below, provides an additional example where the synthesis reaction is a 5′ fill-in reaction. The 5′ overhang is 18 nucleotides for probe A and 17 nucleotides for probe B. For the nucleic acid synthesis reaction the nucleotide mixture contains dCTP-CM-Lys, dGTP, DATP, and dTTP. The 5′ overhang has 7 (probe A) and 10 (probe B) dG residues. Thus in the fill-in reaction, 7 dC-CM-Lys residues (probe A) and 10 dC-CM-Lys residues (probe B) will be inserted into the synthesized nucleic acid. Probe A will contain 7 chelating agent and probe B will contain 10 chelating agents assuming the fidelity of the polymerase to be 100%.

Method of Chelating a Transition Metal Ion to a Nucleic Acid-Chelating Agent Conjugate

Any method known in the art for chelating a transition metal ion to a chelating agent can be utilized in the present invention to chelate a transition metal ion to a nucleic acid-chelating agent conjugate. Typically, the nucleic acid-chelating agent conjugate is mixed with an aqueous solution of the transition metal ion and incubated for a time, usually several minutes (e.g., 5, 10, 15, 20, 30, or 45 minutes) to several hours (e.g., 1, 2, or 3 hours), to form a transition metal-chelating agent-nucleic acid chelate. Following chelation of the transition metal ion, the transition metal-chelating agent-nucleic acid chelate is purified to remove excess transition metal ion. Purification of the transition metal-chelating agent-nucleic acid chelate can be, for example, accomplished though precipitation of the transition metal-chelating agent-nucleic acid chelate using 2% (by weight/volume) lithium perchlorate in acetone or by eluting over a G-25 spin column (Amersham Biosciences, Piscataway, N.J.). The precipitated transition metal-chelating agent-nucleic acid chelate can be resuspended in any suitable buffer, for example 0.01 M sodium phosphate (pH 7.5), for storage or use.

Transition Metal Ions

The transition metal ion is selected based on its ability to coordinately bind to both the chelating agent and to polyhistidine. Examples of transition metal ions include, but are not limited to Ni²⁺, Cu²⁺, Zn²⁺, and Co²⁺. Preferably, the transition metal ion is Ni²⁺.

Nucleic Acid Label

To assist with detection of the nucleic acid, the nucleic acid can be labeled with a radioactive, fluorescent and/or biotin label. The radioactive label can be, for example, a ³H, ³²P, ³³P, or ³⁵S radioactive moiety. Preferably the radioactive label is ³²P. Intensifying screens can be utilized to enhance the level of detection of the radioactive label. The fluorescent label can be for example, a rhodamine, fluorescein, Cy3, or Cy5 fluorescent moiety. The nucleic acid can be labeled on the 5′ or 3′ end of the nucleic acid and/or on a nucleotide within the nucleic acid. Preferably, the label is located on the 5′ end of the nucleic acid. If the nucleic acid is synthesized by a fill-in reaction of two oligonucleotides of uneven length, the label is preferably located on the shorter oligonucleotide. Alternatively, the label can be incorporated with a labeled-nucleotide as the nucleic acid is synthesized. Examples of such labeled-nucleotides include, but are not limited to, Cy5-dCTP, fluorescein-12-dATP, fluorescein-12-dCTP, fluorescein-12-dGTP, fluorescein-12-dTTP, fluorescein-12-ATP, fluorescein-12-CTP, fluorescein-12-GTP, fluorescein-12-TTP, 5′-[α-³⁵S]-dATP, 5′-[α-³⁵S]-dCTP, 5′-[α-³⁵S]-dGTP, 5′-[α-³⁵S]-dTTP, 5′-[α³⁵S]-ATP, 5′-[α-³⁵S]-CTP, 5′-[α-³⁵S]-GTP, 5′-[α-³⁵S]-TTP, 5′-[α³²P]-dATP, 5′-[α³²P]-dCTP, 5′-[α³²P]-dGTP, 5′-[α³²P]-dTTP, 5′-[α³²P]-ATP, 5′-[α³²P]-CTP, 5′-[α³²P]-GTP, and 5′[α³²P]-TTP.

Detecting Polyhistidine-Containing Recombinant Proteins

A polyhistidine-containing recombinant protein can be detected using the above described radioactively- or fluorescently-labeled transition metal-chelating agent-nucleic acid chelate. The transition metal-chelating agent-nucleic acid chelate can also be labeled with biotin, and a polyhistidine-containing recombinant protein can be detected using an enzyme-streptavidin conjugate. Examples of enzymes suitable for use include, but are not limited to horseradish peroxidase (HRP) and alkaline phosphatase (AP). The polyhistidine-containing recombinant protein can be detected by, for example, conjugating a transition metal-chelating agent-nucleic acid chelate to the polyhistidine-containing protein and detecting the conjugate. The transition metal-chelating agent-nucleic acid chelate can be labeled with a radioactive or fluorescent moiety to allow visualization of the conjugated polyhistidine-containing protein.

A polyhistidine-containing recombinant protein also can be detected using a single stranded transition metal-chelating agent-nucleic acid chelate and visualized by utilizing a complementary single stranded nucleic acid probe labeled with a radioactive moiety, a fluorescent moiety, or a biotin moiety (detected by an enzyme-streptavidin conjugate).

His-tag Amplification

Alternatively, the transition metal-chelating agent-nucleic acid chelate can be detected, for example, by a method termed “His-tag amplification.” His-tag amplification includes the steps of amplifying the nucleic acid portion of the transition metal-chelating agent-nucleic acid chelate (e.g., using PCR or real-time PCR) and detecting the amplified nucleic acid. See U.S. Pat. No. 5,665,539 for a general description of using nucleic acid amplification as a means for detection. Amplified nucleic acid can be detected using techniques well known in the art. For example, nucleic acid amplified by PCR can be detected by intercalating agents, such as, for example, ethidium bromide, into the nucleic acid and visualizing the dye. Nucleic acid amplified by real-time PCR can be detected by fluorescence from a fluorescent moiety within an amplification primer.

Western Blotting

Following the transfer of resolved proteins from an acrylamide gel to a membrane, for example, a nitrocellulose or PVDF membrane, a polyhistidine-containing recombinant protein can be detected. The polyhistidine-containing protein can be detected by, for example, incubating the membrane with a nucleic acid that contains at least one nucleotide-chelating agent conjugate that has been chelated to a transition metal ion. The nucleic acid is also labeled with a radioactive or fluorescent label to allow visualization of the polyhistidine-containing protein band or the nucleic acid can be labeled with biotin and the polyhistidine-containing recombinant protein detected with an enzyme-streptavidin conjugate.

In Gel Detection

Polyhistidine-containing recombinant proteins from a protein lysate can be detected in an acrylamide gel by incubating the protein lysate with a radioactive- or fluorescent-labeled transition metal-chelating agent-nucleic acid chelate to form a polyhistidine-containing recombinant protein-nucleic acid conjugate prior to electrophoresis through an acrylamide gel. Following electrophoresis, the acrylamide gel can be dried and exposed to detect the radioactive or fluorescent label. Detecting the polyhistidine-containing recombinant protein in the acrylamide gel would greatly reduce the time for detection because the protein would not need to be transferred to a membrane by western blotting procedures, subsequently detected by an anti-polyhistidine antibody or an enzyme-nickel conjugate, and developed to visualize the polyhistidine-containing recombinant protein.

The acrylamide gel can be a native gel or a semi-denaturing gel. A semi-denaturing gel is a gel minus the SDS. The semi-denaturing gels can further comprise urea. Typically, urea is present at a concentration of about 7M.

In Situ Detection

The transition metal-chelating agent-nucleic acid chelate can be diffused into a fixed tissue or cell sample to detect the presence and cellular location of a polyhistidine-containing recombinant protein. Methods to diffuse a transition metal-chelating agent-nucleic acid chelate into a fixed tissue or cell sample include, but are not limited to, sample dehydration, rehydration, and permeation of cellular membranes (see Wilkinson D. G (1992) In Situ hybridization, A Practical Approach (Oxford University Press, Oxford, U.K.)).

In Vivo Detection

Transfection of small nucleic acids into cells is well known in the art. One such method includes lipid-based transfection using a reagent such as Oligofectamine™ (Invitrogen, Carlsbad Calif.). Using such a method, it is possible to transfect the transition metal-chelating agent-nucleic acid chelate of the present invention into a living cell and follow a polyhistidine-containing recombinant protein in the living cell. Thus, protein expression levels and protein localization can be determined using the transition metal-chelating agent-nucleic acid chelate of the present invention without fixing, and thus without killing, the cell.

Protein Footprinting

A polyhistidine-containing recombinant protein chelated to a transition metal-chelating agent-nucleic acid can be used for protein footprinting (see Sheshberadaran et al., PNAS 85:1-5, 1988). The polyhistidine-containing recombinant protein-nucleic acid chelate can be digested with a protease to help identify the solution structure of the polyhistidine-containing recombinant protein. Knowledge of the solution structure will help to further refine a known three dimensional structure of a protein in the presence and absence of a substrate. Subtle conformational changes induced by a substrate binding protein can be detected by protein footprinting.

Protein-Protein Detection

Determination of Affinity Between Two Interacting Partners

The radioactive- or fluorescent-labeled transition metal-chelating agent-nucleic acid chelate described above can be used in an assay to determine affinity between two interacting partners (e.g., protein:protein, protein:nucleic acid, and protein:molecule). See Phizicky E. M and Fields S (1995) Microbiology Reviews 59, 94-123 for a general discussion of affinity determination. One of the interacting partners contains a polyhistidine sequence. The polyhistidine-containing recombinant protein is conjugated to the transition metal-chelating agent-nucleic acid chelate (bearing a detectable label). The other partner is conjugated to biotin. The polyhistidine-containing partner is incubated with varying concentrations of the biotinylated partner. Following incubation, the biotinylated partner is captured by, for example, a streptavidin magnetic bead. The beads can be washed to remove unbound material. The amount of radioactivity or fluorescence in the bound sample can be determined and the count can be correlated to the number of interacting partner complexes present in the sample. Alternatively, His-tag amplification (e.g., real-time PCR) can be used to quantitate the number of interacting partner complexes present in the sample.

Determination of a Protein Motif that is Involved in a Protein-Protein Interface

To determine if a protein motif or amino acid is involved in a protein-protein interface a recombinant protein can be constructed with specific mutations and/or deletions and the level of interaction with protein partners or substrates can be measured as described above. The importance of the motif or a specific amino acid can be determined by the level of interaction of the protein partners. Thus, the three dimensional structure can be refined.

In Situ Protein-Protein Hybridization

To identify the cellular loci of protein-protein interactions, in situ protein-protein hybridization can be used. Data derived from such in situ assays may allow the determination of changes on the level of interaction between a polyhistidine-containing recombinant protein and other proteins following a specific protein modification, such as phosphorylation.

Screening Peptide Ligands

The transition metal-chelating agent-nucleic acid chelate can be used to identify a peptide ligand from a peptide library, for example a randomized peptide library, which binds to a particular protein of interest. The peptide library can be synthesized to include a polyhistidine sequence. The polyhistidine sequence can be located on the amino- or carboxy-terminus of the library peptides. The library peptides can be 1 amino acid or more (i.e., 1, 2, 3, 4, 5, 6, 7, 10, 12, 15, 18, 20, 23, 25, or more amino acids) in length, not including the polyhistidine sequence. The polyhistidine sequence preferably contains 6 histidine residues (e.g., 6×His). A surface, such as, for example, the surface of a well from a multi-well plate, can be coated with the protein of interest. The peptide library can be added to the wells and incubated for a time, usually 1 to 4 hours (i.e., 1, 2, 3, or 4 hours) or more to allow the library peptides time to bind to the protein of interest. The wells can be washed to remove unbound library peptides. A transition metal-chelating agent-nucleic acid chelate can be added to the wells and the transition metal-chelating agent-nucleic acid chelate will bind to the polyhistidine sequences of the bound library peptides. The wells can be washed to remove any unbound transition metal-chelating agent-nucleic acid chelate. The bound transition metal-chelating agent-nucleic acid chelate can be detected as described above (i.e., radioactive, fluorescent, biotin, or His-tag amplification). Signal detection (i.e., radioactivity, fluorescence, or amplified nucleic acid) indicates that a peptide from the peptide library has bound to the protein. Peptide identification can be determined using techniques well known in the art. For example, successively smaller pools of peptides can be used to identify the peptide or peptides that bind to the protein.

Screening for Proteins that Bind a Known Peptide Ligand

The transition metal-chelating agent-nucleic acid chelate can be used to identify candidate biomolecules (i.e., protein, nucleic acid, and small molecules) in a biomolecule mixture that can bind a peptide ligand. The biomolecule mixture, for example a cell or tissue extract, can be resolved and transferred to a solid support, for example a nitrocellulose or PVDF membrane, to create an immobilized biomolecule mixture. The biomolecule mixture can be resolved using well known techniques for resolving biomolecule, for example, gel electrophoresis, isoelectric focusing, or column chromatography. The peptide ligand can be synthesized to include a polyhistidine sequence. The polyhistidine sequence can be located on the amino- or carboxy terminus of the library peptides. The polyhistidine sequence preferably contains 6 histidine residues (e.g., 6×His). The peptide can be incubated with the immobilized biomolecule mixture. The immobilized biomolecule mixture can be washed to remove any unbound peptide. A transition metal-chelating agent-nucleic acid chelate can be added to the immobilized biomolecule mixture and incubated for a time to allow the transition metal-chelating agent-nucleic acid chelate time to chelate any bound peptides having the polyhistidine sequence. The immobilized biomolecule mixture can be washed to remove any unbound transition metal-chelating agent-nucleic acid chelate. The bound transition metal-chelating agent-nucleic acid chelate can be detected as described above (i.e., radioactive, fluorescent, biotin, or His-tag amplification).

Alternatively, the peptide ligand can be incubated with the biomolecule mixture to allow a complex to form between the peptide ligand and any biomolecule or biomolecules present in the mixture that can bind the peptide ligand. The biomolecules in the mixture then can be resolved and transferred to a solid support, for example a nitrocellulose or PVDF membrane, to create an immobilized biomolecule mixture. A transition metal-chelating agent-nucleic acid chelate can be added to the immobilized biomolecule mixture and incubated for a time to allow the transition metal-chelating agent-nucleic acid chelate time to chelate the polyhistidine sequence. The immobilized biomolecule mixture can be washed to remove any unbound transition metal-chelating agent-nucleic acid chelate. The bound transition metal-chelating agent-nucleic acid chelate can be detected as described above (i.e., radioactive, fluorescent, biotin, or His-tag amplification).

Diagnostics

Apatamers

Nucleic acid apatamers that recognize specific cell surfaces or a specific receptors on a specific cell type have been developed (U.S. Pat. No. 5,475,096 and U.S. Pat. No. 6,344,321). Such nucleic acids may be used to transport a therapeutic or prophylactic drug or protein to the specific cell type. The nucleic acid apatamer can be synthesized to include at least one nucleotide having covalently bonded thereto a chelating agent. Such an apatamer can be used to chelate a transition metal ion and coordinately be attached to a therapeutic or prophylactic polyhistidine-containing drug or protein through the transition metal ion interaction. The apatamer will direct the therapeutic or prophylactic drug to the cell of interest.

Determination of Polyhistidine Tag Removal

Following purification of a polyhistidine-containing recombinant protein, the polyhistidine-containing moiety can be removed by a protease, provided a cleavage site was engineered into the protein sequence. To verify removal of the polyhistidine moiety a radioactive- or fluorescent-labeled transition metal-chelating agent-nucleic acid chelate can be used. Using a nitrocellulose based assay, the amount of his-tag can be determined (Jellinik et al., (1993) Proc. Natl. Sci. U.S.A. (90) 11227-11231).

Quantification

The amount of a polyhistidine-containing recombinant protein in a cellular or tissue lysate can be quantitated using a radioactive- or fluorescent-labeled transition metal-chelating agent-nucleic acid chelate. The protein lysate can be immobilized to a solid support such as an ELISA plate or spotted onto a membrane, for example nitrocellulose or PVDF. The radioactive- or fluorescent-labeled transition metal-chelating agent-nucleic acid chelate can be incubated with the immobilized protein lysate. The immobilized protein can be washed extensively to remove unbound radioactive- or fluorescent-labeled transition metal-chelating agent-nucleic acid chelate. The level of radioactivity or fluorescence can be determined and the level correlates to the amount of polyhistidine-containing protein in the sample.

Greater sensitivity can also be achieved by amplifying the nucleic acid sequence of the transition metal-chelating agent-nucleic acid chelate, for example by His-tag amplification (described above), and detecting the amplified nucleic acid. For example, the His-tag amplification can include real-time PCR, and the amount of transition metal-chelating agent-nucleic acid chelate present in the starting material can be quantitated.

For detection of a small amount of polyhistidine-containing recombinant protein or a large volume of lysate, a radioactive-labeled- or fluorescently-labeled-transition metal-chelating agent-nucleic acid chelate can be added directly to the lysate. Following an incubation period, usually 1 hour with gentle mixing, the sample can be filtered on a membrane that interacts only with protein and not nucleic acid (e.g., PVDF). Thus, any nucleic acid that is associated with a polyhistidine tag can be detected. The amount of polyhistidine-containing recombinant protein can be deduced from the radioactive count or fluorescent emission of the bound material.

Nucleic acid sequences can also be detected using a transition metal-chelating agent-nucleic acid chelate. Following a Southern or northern blot, a transition metal-chelating agent-nucleic acid chelate probe can be generated and hybridized using the teachings herein and methods well known in the art. See for example, Sambrook et al., MOLECULAR CLONING: A LABORATORY MANUAL, 2d ed., 1989 and Ausubel et al., CURRENT PROTOCOLS IN MOLECULAR BIOLOGY, John Wiley & Sons, New York, N.Y., 1989 for general protocols for Southern blotting, northern blotting and hybridization. The hybridized transition metal-chelating agent-nucleic acid chelate probe can be detected, for example, using a polyhistidine-containing enzyme (e.g., polyhistidine tagged AP, HRP, and P-galactosidase).

All patents patent applications and references cited in this application are incorporated herein by reference in their entirety.

The following examples are offered by way of illustration and do not limit the invention disclosed herein.

### EXAMPLES

### Example 1

Synthesis of dCTP-CM-Lys

dCTP-CM-Lys was synthesized using the following reaction scheme.

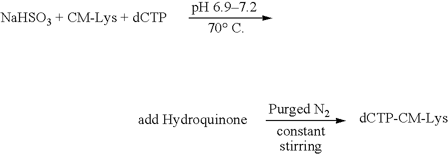

Formation of the dCTP-CM-Lys was monitored by HPLC using an analytical C-18 column. The HPLC solvents were as follows:

Solvent A:

5 mM TBAP (tetrabutyl ammonium phosphate) in 60 mM NH₄H₂PO₄ (pH 5).

Solvent B:

5 mM TBAP in methanol.
dCTP-CM-Lys was purified over a DEAE-Sephadex A-25 column using an ionic gradient of 0.1M to 1M of TEAB (triethylammonium bicarbonate buffer pH 7.0-7.5). Fractions were pooled, dried over a Rotovapor and washed with ethanol (3×). Mass spectrophotometer analysis (M-Scan Inc.) confirmed the synthesis of dCTP-CM-Lys. The fraction pool of dCTP-CM-Lys was fractionated over an analytical C-18 column in order to quantitate the purity of the sample.

### Example 2

Incorporation of dCTP-CM-Lys into a Nucleic Acid by a fill-in Reaction of Annealed Oligonucleotides of Unequal Length

Klenow fragment of E. coli DNA polymerase I and Taq DNA polymerase were used to determine if dCTP-CM-Lys could be inserted into a synthesized nucleic acid. The sequences of the primer/template substrates that were employed are shown below and are denoted as substrates A and B.

Label the 5′-end of an Oligonucleotide

Primer sequence—^{5′}CCAACCACACCACACCG^{3′} (SEQ ID NO:1) was labeled on the 5′ end with a T₄ kinase reaction. The kinase reaction was assembled as follows:
2 μL primer (500 μM)
2 pLyATP (0.66 μM)
4 μL 5×Kinase buffer
2 μL T₄ kinase
10 μL H₂O

The reaction mix was incubated at 37° C. for 15 minutes. The ratio of primer: γATP was 750:1 so as to consume all the radioactive ³²P in the reaction and eliminate a purification step. Following the kinase reaction, the mix was incubated at 100° C. for 1 minute, to denature the T₄ kinase.

Primer/Template DNA Polymerase Substrates

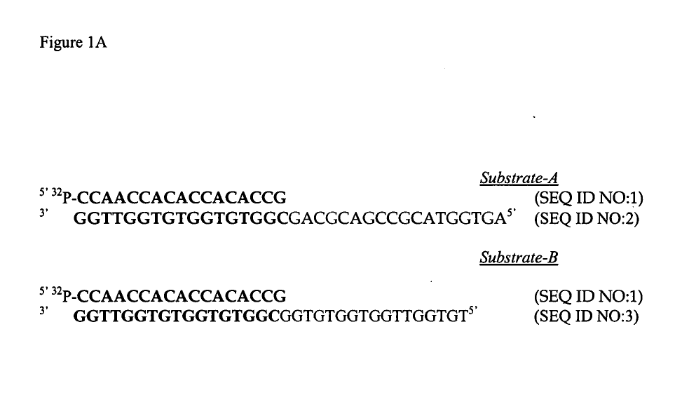

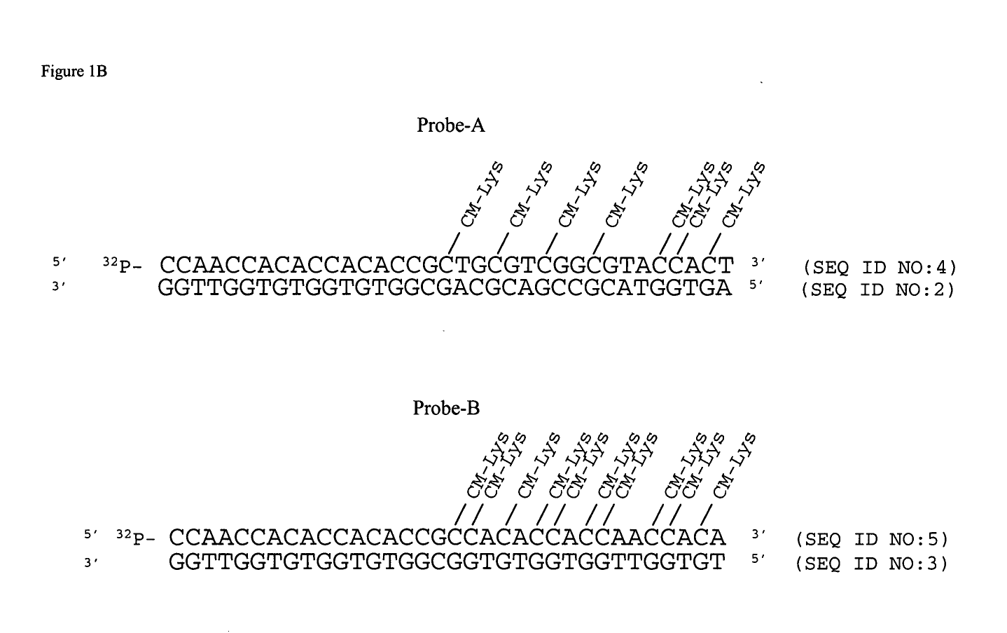

The primer (SEQ ID NO:1) and the template (SEQ ID NOS:2 or 3) were mixed in a ratio of 1:5 (50 μM primer and 250 μM template) and heated to 100° C. for 1 minute to remove any secondary structure in the nucleic acid. The primer/template mixtures were gradually cooled to room temperature. The primer/template mixtures were incubated at room temperature for 2 hours to allow the primer to anneal to the template. The resulting annealed primer/template pairs are shown in FIG. 1A.

DNA Polymerase Assay

The 5′ overhang was filled in using a Klenow DNA polymerase or Taq DNA polymerase. The reactions were assembled as follows

| | Concentration in | |
|---|---|---|
| Reaction component | reaction mix | |
| labeled primer/template | 2.8 | μM |
| dCTP-CM-Lys | 200 | μM |
| dGTP, dATP, dTTP | 100 | μM each |
| MgCl₂ | 2 | mm |

| | |
|---|---|
| buffer | 1X |

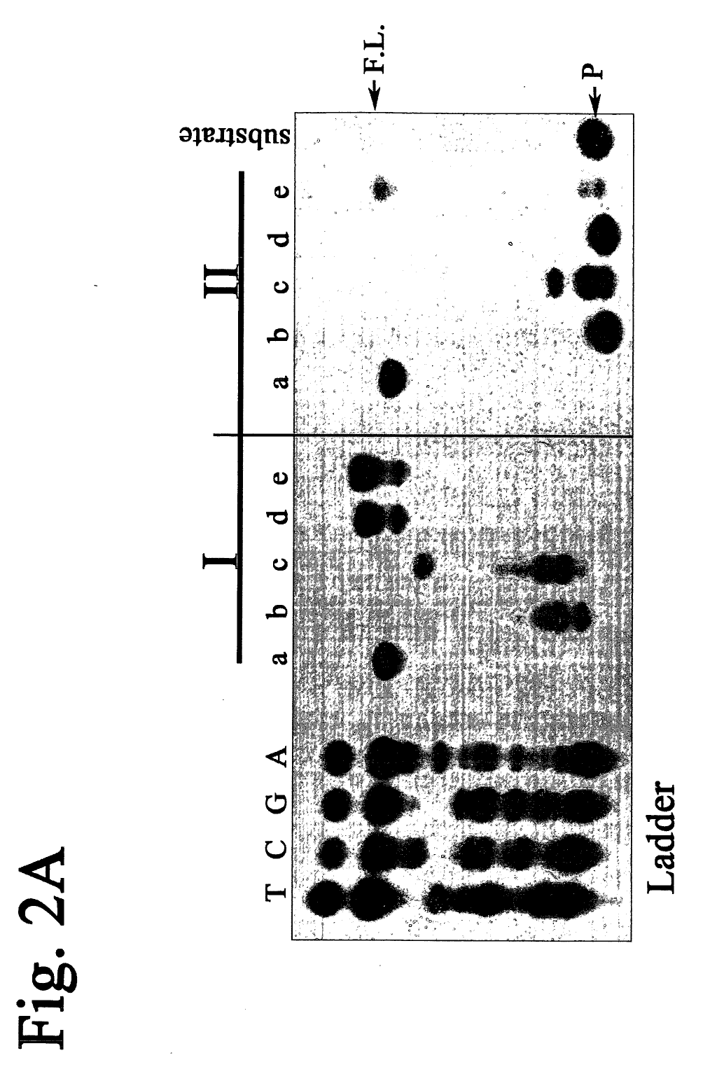

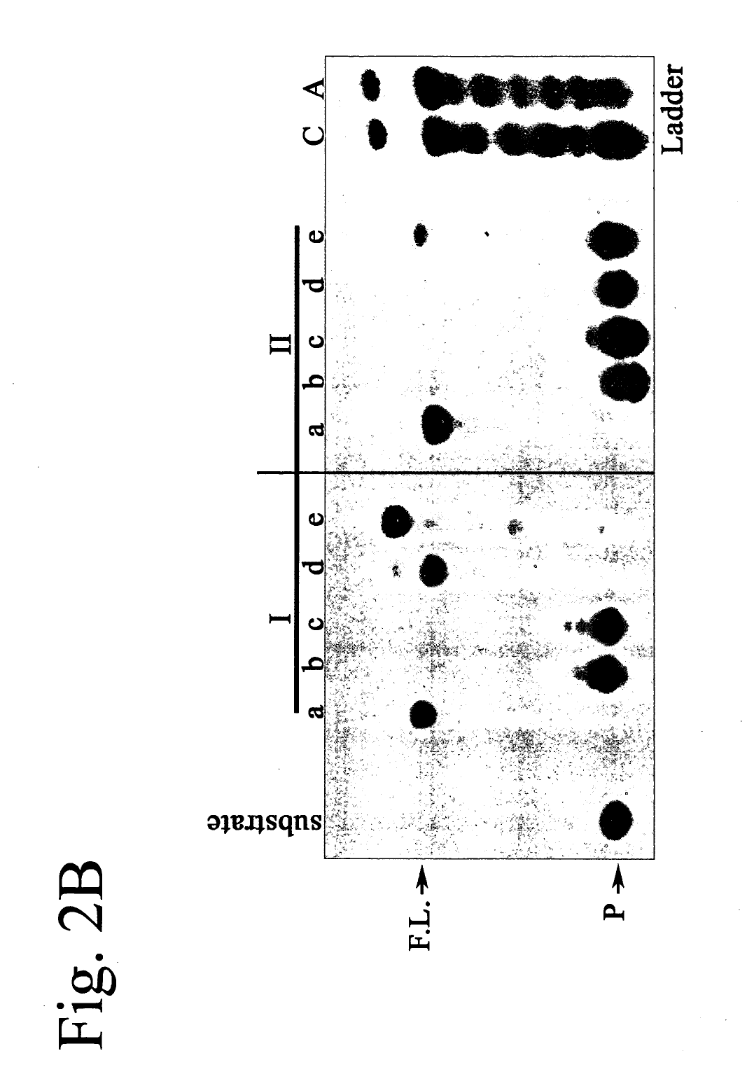

Following assembly, the reaction mix was incubated at 37° C. for 1 hour to allow fill-in of the 5′ overhang. See FIG. 1B for the product of the fill-in reaction. The locations of the chelating agents are shown in the nucleic acid as shaded nucleotides. Synthesis of full length nucleic acid was verified by gel electrophoresis. FIGS. 2A and 2B show synthesis of full length nucleic acid for substrate 1 and substrate 2, respectively. Post-nucleic acid synthesis, the Klenow fragment was inactivated by incubating the reaction at 100° C. for 1 minute. The incorporated chelating agent was charged with a transition metal ion, Ni²⁺, in an overnight reaction with a solution of nickel sulfate to form a transition metal ion-chelating agent-nucleic acid chelate. The transition metal ion-chelating agent-nucleic acid chelate was precipitated using a 2% lithium perchlorate in acetone, and resuspended in 0.01 M NaPi (pH 7.5) buffer to a final concentration of 450 nM in the primer strand termini.

### Example 3

Detection of a Polyhistidine-Containing Protein on a Nitrocellulose Membrane

SDS PAGE and Western Blotting

His-tagged β-gal (110 kDa) and BLV-1 (35 kDa) were resolved over a 4%-20% SDS denaturing protein gel (BioRad). The concentration of the protein samples ranged from 3 ng-2 μg per lane. As a control, 2 μg and 400 ng of the wild-type β-gal protein samples (containing no His-tag) were also loaded. Following electrophoresis, the protein bands were transferred to a nitrocellulose membrane, per the usual western blotting protocol. The membrane was blocked using 20 mL 1×PBS buffer containing 500 mg sperm herring DNA for 1 hour at room temperature.

Protein Detection

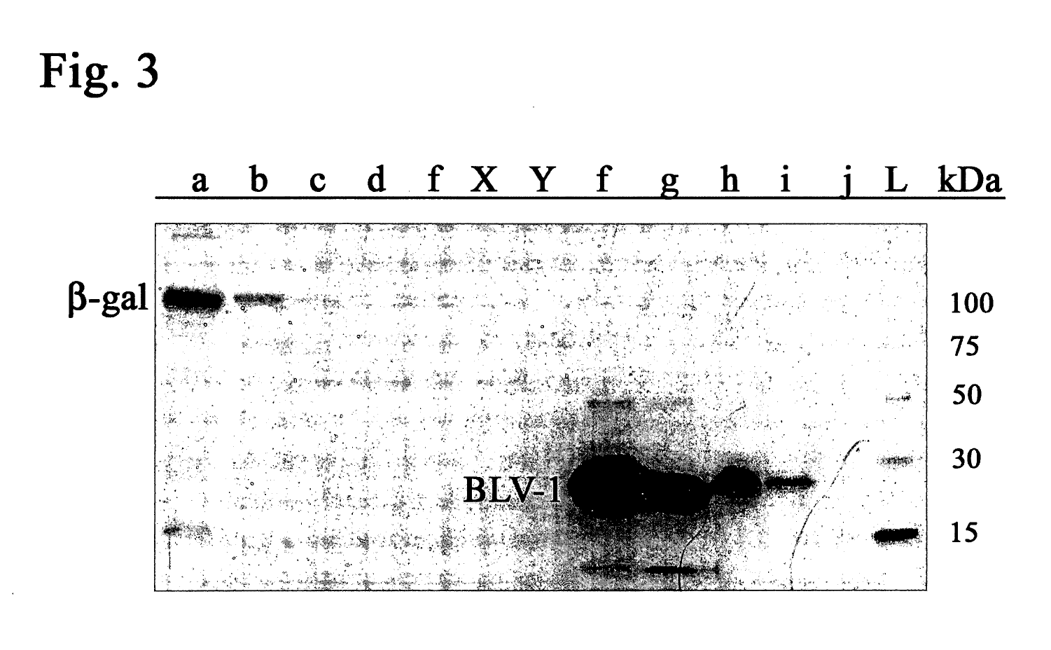

An oligo-probe (Probe B) that was labeled with ³²P, as described above, was added to the blocking mix (to a final concentration of 45 pM) and was incubated with gentle swirling, 2-12 hours at room temperature. Finally the membrane was washed with 20 mL blocking solution (PBS+sperm herring DNA) by gentle mixing 30 minutes. The protein bands were detected by exposing the membrane film. See FIG. 3 for an autoradiogram.

### Example 4

Conjugating α-N,N-bis-Carboxymethyl Lysine with an NHS Modified Oligonucleotide

Oligo(dT)₁₀ containing an NHS modified 5′-terminus was synthesized by Midland Certified Reagent Company (Midland Tex.). The oligo(dT) was prepared with phosphorothioate nucleotides. The oligo(dT) was delivered attached to the CPG beads used during synthesis.

A 20 μmol solution of α-N,N-bis-carboxymethyl lysine was prepared in a 1:0.75:0.2 mixture of DMSO:H₂O:triethylamine, pH 8. Using two 1 ml syringes, the α-N,N-bis-carboxymethyl lysine solution was gently added to a chamber containing the oligo(dT). The reaction was incubated for 4 hours at room temperature with gentle shaking. Following incubation, the oligo(dT) was washed 2 times with 1 ml H₂O and incubated in 1 ml NH₄OH for 30 minutes to remove the CPG bead. The nucleic acid solution was dried under vacuum and resuspended in H₂O. Mass spectra, using MALDI-MS, confirmed that α-N,N-bis-carboxymethyl lysine was conjugated to the oligonucleotide.

1. A nucleic acid having covalently bonded to at least one nucleotide of the nucleic acid, a chelating agent, the covalently bonded chelating agent having an affinity for a transition metal ion.

2. The nucleic acid of claim 1 wherein the nucleic acid comprises a plurality of covalently bonded chelating agents.

3. The nucleic acid of claim 1 wherein the nucleic acid is chelated to a transition metal ion.

4. The nucleic acid of claim 3 wherein the transition metal ion is selected from the group consisting of Ni²⁺, Cu²⁺, Zn²⁺, and Co²⁺.

5. The nucleic acid of claim 4 wherein the transition metal ion is Ni²⁺.

6. The nucleic acid of claim 1 wherein the nucleic acid is labeled with a radioactive moiety.

7. The nucleic acid of claim 6 wherein the radioactive moiety is selected from the group consisting of ³²P, ³³P, ³⁵S, and ³H.

8. The nucleic acid of claim 6 wherein the radioactive moiety is ³²P and the ³²P moiety is a 5′ label or a 3′ label.

9. The nucleic acid of claim 1 wherein the nucleic acid is labeled with a fluorescent moiety.

10. The nucleic acid of claim 1 wherein the nucleic acid is labeled with a biotin moiety.

11. A method of generating a nucleic acid having covalently bonded to at least one nucleotide of the nucleic acid, a chelating agent, the covalently bonded chelating agent having an affinity for a transition metal ion, the method comprises the steps of:
a. determining which nucleotides in a nucleic acid will be covalently bonded to the chelating agent; and
b. synthesizing the nucleic acid utilizing a nucleotide having covalently bonded thereto a chelating agent determined in step (a).

12. The method of claim 11 wherein the nucleic acid in step (b) is synthesized by an enzymatic reaction.

13. The method of claim 12 wherein the enzymatic reaction utilizes an enzyme selected from the group consisting of a DNA polymerase, a PCR polymerase, an RNA polymerase, a reverse transcriptase, and mutants, variants, and derivatives thereof.

14. The method of claim 13 wherein the enzyme is a DNA polymerase and the DNA polymerase is derived from a mesophilic organism.

15. The method of claim 14 wherein the DNA polymerase derived from a mesophilic organism is selected from the group consisting of E. coli DNA polymerase I (proficient or deficient in 3′→5′ exonuclease activity), T4 DNA polymerase, and mutants, variants, and derivatives thereof.

16. The method of claim 13 wherein the enzyme is a PCR polymerase and the PCR polymerase is a thermostable polymerase.

17. The method of claim 16 wherein the thermostable polymerase is selected from the group consisting of Taq, Tne, Tma, Tth, Pfu, VENT™, DEEPVENT™, pfx™, and mutants, variants and derivatives thereof.

18. The method of claim 12 wherein the enzymatic reaction is PCR.

19. The method of claim 12 wherein the nucleic acid is synthesized utilizing a nucleotide having covalently bonded thereto a chelating agent.

20. The method of claim 1 wherein the nucleic acid in step (b) is synthesized by a chemical reaction.

21. The method of claim 20 wherein the chemical reaction uses phosphoroamidite chemistry.

22. The method of claim 20 wherein the chemical reaction utilizes an automated oligonucleotide synthesizer.

23. A method of generating a nucleic acid having covalently bonded to at least one nucleotide of the nucleic acid, a chelating agent, the covalently bonded chelating agent having an affinity for a tmmsition metal ion, the method comprises the steps of:
a. providing the nucleic acid; and
b. bonding the chelating agent to the nucleic acid with a crosslinking agent.

24. A nucleotide-chelating agent conjugate comprising a nucleotide having covalently bonded thereto a chelating agent, the covalently bonded chelating agent having an affinity for a transition metal ion.

25. The nucleotide of claim 24 wherein the nucleotide is a deoxyribonucleotide.

26. The deoxyribonucleotide of claim 24 wherein the deoxyribonucleotide is selected from the group consisting of dCTP, dATP, dGTP, dTTP, dITP and derivatives and analogs thereof.

27. The deoxyribonucleotide of claim 26 wherein the deoxyribonucleotide is dCTP.

28. The nucleotide of claim 24 wherein the nucleotide is a ribonucleotide.

29. The ribonucleotide of claim 27 wherein the ribonucleotide is selected from the group consisting of CTP, ATP, GTP, UTP and derivatives and analogs thereof.

30. The nucleotide of claim 24 wherein the chelating agent is NTA.

31. The nucleotide of claim 30 wherein the NTA is α-N,N-bis-carboxymethyl lysine.

32. The nucleotide of claim 24 wherein the transition metal ion is selected from the group consisting of Ni²⁺, Cu²⁺, Zn²⁺, and Co²⁺.

33. The nucleotide of claim 32 wherein the transition metal ion is Ni²⁺.

34. A method of synthesizing a nucleotide-chelating agent conjugate, the method comprises the step of covalently bonding a chelating agent to a nucleotide to form the nucleotide-chelating agent conjugate, the covalently bonded chelating agent having an affinity for a transition metal ion.

35. The method of claim 34 wherein the step of coupling is enzymatic coupling.

36. The method of claim 35 wherein the enzymatic coupling utilizes an enzyme selected from the group consisting of pyrophosphatase, terminal nucleotidyl transferase, recombinase, ligase, isomerase, and a ribozyme.

37. The method of claim 34 wherein the step of coupling is chemical coupling.

38. The method of claim 37 wherein the chelating agent is NTA.

39. The method of claim 38 wherein the NTA is α-N,N-bis-carboxymethyl lysine.

40. A method of chelating a transition metal ion to a nucleic acid having covalently bonded to at least one nucleotide of the nucleic acid, a chelating agent, the covalently bonded chelating agent having an affinity for a transition metal ion, the method comprises the steps of:
a. mixing an excess of a transition metal ion and the nucleic acid to form a mixture;
b. incubating the mixture for a time to form a transition metal-chelating agent-nucleic acid chelate; and
c. purifying the transition metal-chelating agent-nucleic acid chelate from the excess transition metal ion.

41. The method of claim 40 wherein step (c) is performed by precipitation using 2% lithium perchlorate.

42. A method for detecting a polyhistidine-containing recombinant protein wherein the method comprises the steps of
a. forming a conjugate of a transition metal-chelating agent-nucleic acid chelate with the polyhistidine-containing recombinant protein; and
b. detecting the conjugate.

43. The method of claim 42 wherein the polyhistidine recombinant protein to be detected is present in a gel.

44. The method of claim 43 wherein the step of forming the conjugate is performed prior to resolving the protein mixture on the gel.

45. The method of claim 42 wherein the gel is selected from the group consisting of a semi-denaturing gel and a native gel.

46. The method of claim 45 wherein the gel is a semi-denaturing gel and the semi-denaturing gel further comprises 7M urea.

47. The method of claim 42 wherein the recombinant protein to be detected has been transferred to a membrane.

48. The method of claim 42 wherein the chelating agent is NTA.

49. The method of claim 48 wherein the NTA is α-N,N-bis-carboxymethyl lysine.

50. The method of claim 42 wherein the transition metal-chelating agent-nucleic acid further comprises a label.

51. The method of claim 50 wherein the label is a radioactive label.

52. The method of claim 50 wherein the label is a fluorescent label.

53. The method of claim 50 wherein the label is a biotin label.

54. The method of claim 42 wherein the step of detecting the conjugate comprises His-tag amplification.

55. A method for His-tag amplification of a transition metal-chelating agent-nucleic acid chelate, the method comprises the step of amplifying the nucleic acid portion of the chelate.

56. The method of claim 55 further comprising the step of detecting the amplified nucleic acid.

57. The method of claim 55 wherein the step of amplifying the nucleic acid portion of the chelate comprises PCR.

58. The method of claim 55 wherein the step of amplifying the nucleic acid portion of the chelate comprises real-time PCR.

59. A method for identifying a peptide ligand that binds a biomolecule, wherein the peptide ligand is identified from a peptide library, the method comprises the steps of:
(a) immobilizing the biomolecule;
(b) contacting the biomolecule with a peptide library, wherein the peptide library comprises peptides having a polyhistidine sequence;
(c) forming a conjugate of a transition metal-chelating agent-nucleic acid chelate with the polyhistidine sequence of the library peptides; and
(d) detecting the chelate.

60. The method of claim 59 wherein the step of immobilizing the biomolecule comprises immobilizing the biomolecule to a surface.

61. The method of claim 60 wherein the surface comprises the surface of a well of a multi-well plate.

62. The method of claim 59 wherein the step of detecting comprises His-tag amplification.

63. The method of claim 62 wherein the His-tag amplification includes real-time PCR.

64. The method of claim 59 wherein the chelate further comprises a moiety selected from the group consisting of a radioactive moiety, a fluorescent moiety, and biotin.

65. A method for identifying a biomolecule that can bind to a peptide ligand, the method comprises the step of:
(a) providing a biomolecule mixture;
(b) resolving the biomolecule mixture;
(c) immobilizing the biomolecule mixture;
(d) contacting the biomolecule mixture with a peptide library, wherein the peptide library comprises peptides having a polyhistidine sequence;
(e) forming a conjugate of a transition metal-chelating agent-nucleic acid chelate with the polyhistidine of the peptides; and
(f) detecting the chelate.

66. The method of claim 65 wherein the step of detecting comprises His-tag amplification.

67. The method of claim 66 wherein the His-tag amplification includes real-time PCR.

68. The method of claim 65 wherein the peptide further comprises a moiety selected from the group consisting of a radioactive moiety, a fluorescent moiety, and biotin.

69. A method for identifying a biomolecule that can bind to a peptide ligand, the method comprises the steps of:
(a) providing a biomolecule mixture;
(b) contacting the biomolecule with a peptide library, wherein the peptide library comprises peptides having a polyhistidine sequence;
(c) resolving the biomolecule mixture;
(d) immobilizing the biomolecule mixture;
(e) forming a conjugate of a transition metal-chelating agent-nucleic acid chelate with the polyhistidine of the peptides; and
(f) detecting the chelate.

70. The method of claim 69 wherein the step of detecting comprises His-tag amplification.

71. The method of claim 70 wherein the His-tag amplification includes real-time PCR.

72. The method of claim 69 wherein the peptide further comprises a moiety selected from the group consisting of a radioactive moiety, a fluorescent moiety, and biotin.


SEQUENCE LISTING (5 sequences)

SEQ ID NO: 1 - DNA, 17 bp, Artificial
  misc_feature: Primer for template A and B
ccaaccacaccacaccg

SEQ ID NO: 2 - DNA, 35 bp, Artificial
  misc_feature: Template A
ggttggtgtggtgtggcgacgcagccgcatggtga

SEQ ID NO: 3 - DNA, 34 bp, Artificial
  misc_feature: Template B
ggttggtgtggtgtggcggtgtggtggttggtgt

SEQ ID NO: 4 - DNA, 35 bp, Artificial
  misc_feature: Product from fill-in reaction
ccaaccacaccacaccgctgcgtcggcgtaccact

SEQ ID NO: 5 - DNA, 34 bp, Artificial
  misc_feature: Product from fill-in reaction
ccaaccacaccacaccgccacaccaccaaccaca